(19) United States
(12) Patent Application Publication
(10) Pub. No.: US 2010/0297456 A1
(43) Pub. Date: Nov. 25, 2010

(54) Diamine, Polyamic Acid and Polyimide

(75) Inventors: Takanori Tano, Chiba (JP); Koei Suzuki, Kanagawa (JP); Yusuke Tsuda, Fukuoka (JP)

Correspondence Address: COOPER & DUNHAM, LLP, 30 Rockefeller Plaza, 20th Floor, NEW YORK, NY, 10112, US

(73) Assignee: RICOH COMPANY, LTD., Tokyo (JP)

(21) Appl. No.: 12/786,061

(22) Filed: May 24, 2010

(30) Foreign Application Priority Data
May 25, 2009 (JP) 2009-125634

(51) Int. Cl. C08G 63/685; C07C 229/42; B32B 27/00

(52) U.S. Cl. 428/473.5; 560/45; 528/184

(57) ABSTRACT

Disclosed is a diamine represented by a general formula of:

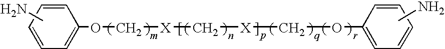

wherein X is an ester bond, each of m and n is independently a natural number, each of p and r is independently 0 or 1, q is an integer of 0 or more, a sum of m, n, and q is 20 or less, and when q is 0, p is 1 and r is 0.

### BACKGROUND OF THE INVENTION

1. Field of the Invention

One aspect of the present invention relates to at least one of a diamine, a polyamic acid, a polyimide, a composition, a laminated structure, an electronic element array, an image display medium, and an image display device.

2. Description of the Related Art

Conventionally, polylactic acid, polycaprolactone and the like have been known as biodegradable plastics but none of polyamic acids and polyimides having biodegradability has been known. Generally, a polyamic acid and a polyimide are obtained by causing ring-opening and addition polymerization of a diamine and a tetracarboxylic dianhydride.

Herein, an aliphatic diamine obtained by causing a dehydration reaction of glycine or phenylalanine with 2-aminoethanol is known for a diamine having biodegradability (see International Publication No. 99/11703).

However, such an aliphatic diamine has a problem that it may be difficult to cause a ring-opening and addition polymerization reaction with tetracarboxylic dianhydride in good yield because its amino group has a strong basicity.

Meanwhile, an organic thin-film transistor using an organic semiconductor material has actively been studied recently. As examples of benefits of use of an organic semiconductor material, there are provided a high flexibility, a possibility of attaining a large surface area, a possibility of simplifying a production process, an inexpensive production apparatus, and the like.

An on-off current ratio has been used as a parameter indicating the characteristic of an organic thin-film transistor. For an organic thin-film transistor, an on-state current I_{ds} flowing between source and drain electrodes in a saturation region is represented by a formula of:

I_{ds}=μ Cᵢₙ W(V_{G}−V_{TM})²/2L

(in which formula, μ is a field-effect mobility, Cᵢₙ is a capacitance per unit area of a gate insulating film and is represented by a formula of:

Cin=ε δ₀/d

(in which formula, ε is a relative dielectric constant of the gate insulating film, ε₀ is a dielectric constant of vacuum, and d is a thickness of the gate insulating film.), W is a channel width, L is a channel length, V_{G} is a gate voltage, and V_{TH} is a threshold voltage.). From this formula, it is understood that it is effective to increase μ, to decrease L, and to increase W, in order to increase the on-state current. Herein, μ greatly depends on the characteristic of an organic semiconductor material. On the other hand, L and W are attributed to the structure of an organic thin-film transistor. Additionally, the distance between source and drain electrodes are generally decreased in order to decrease L but an organic thin-film transistor has a small μ whereby it is desired that L is 10 μm or less and preferably 5 μm or less.

It is desired that an ink jet printing method is used to form such a source and drain electrode pattern. When an ink jet printing method is used, it is possible to write a pattern directly whereby the efficiency of using a material is high and it is possible to attain simplification and cost reduction of a production process. However, it may be difficult for an ink jet printing method to attain a small amount of ejection, and it may be difficult to form a pattern with a size of 30 μm or less when a landing precision dependent on a mechanical error or the like is taken into consideration.

Thus, a method is known in which a wettability changing layer containing a material whose surface free energy is capable of being changed by ultraviolet ray irradiation is irradiated with ultraviolet rays so as to change the surface free energy and subsequently a source and drain electrode pattern is formed on the wettability changing layer by using an ink jet printing method. However, even if a high power ultraviolet ray lamp is used, a long period of time may be needed for irradiation, and accordingly, a tact time may be long, whereby there is a problem such that it may be impossible to attain simplification or cost reduction of a production process. Furthermore, there is a problem such that the insulating property of the wettability changing layer may be degraded due to ultraviolet ray irradiation for changing the surface free energy.

Hence, Japanese Patent Application Publication No. 2008-227294 discloses a laminated structure which includes a wettability changing layer that contains a soluble polyimide having a side chain and containing two or more portions at which cleavage of bonding is caused by ultraviolet ray absorption, and a patterned electrically conductive layer formed on the wettability changing layer.

### SUMMARY OF THE INVENTION

According to one aspect of the present invention, there is provided a diamine represented by a general formula of:

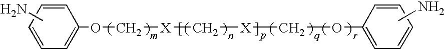

wherein X is an ester bond, each of m and n is independently a natural number, each of p and r is independently 0 or 1, q is an integer of 0 or more, a sum of m, n, and q is 20 or less, and when q is 0, p is 1 and r is 0.

### BRIEF DESCRIPTION OF THE DRAWINGS

FIG. 1 is a cross-sectional diagram illustrating one example of a laminated structure according to an embodiment of the present invention.

FIGS. 2A and 2B are diagrams illustrating one example of an electronic element array according to an embodiment of the present invention.

FIG. 3 is a cross-sectional diagram illustrating one example of an image display medium according to an embodiment of the present invention.

FIG. 4 is a diagrammatic perspective view illustrating one example of an image display device according to an embodiment of the present invention.

FIG. 5 is a diagram illustrating a ¹H-NMR spectrum of a diamine in a practical example of the present invention.

FIG. 6 is a diagram illustrating a change in the weight of a film in any of a practical example of the present invention and comparative examples versus a period of time of leaving in compost.

FIGS. 7A and 7B are photographs illustrating films in practical example 1 and comparative example 2 before leaving in compost and after leaving in compost for 30 days.

### DETAILED DESCRIPTION OF THE PREFERRED EMBODIMENTS

Next, at least one embodiment of the present invention will be described in conjunction with the accompanying drawings(s).

A diamine according to an embodiment of the present invention is represented by a general formula of

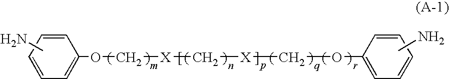

(in which formula, X is an ester bond, each of m and n is independently a natural number, each of p and r is independently 0 or 1, and q is an integer of 0 or more, wherein the sum of m, n, and q is 20 or less and preferably 10 or less. Herein, when q is 0, p is 1 and r is 0.). Hence, a diamine according to an embodiment of the present invention may have biodegradability, because it may be possible to cause a ring-opening and addition polymerization reaction with a tetracarboxylic dianhydride in good yield. However, a diamine in which the sum of m, n, and q is more than 20 in general formula (A-1) may have a reduced solubility in an aprotic polar solvent. Such an organic aprotic solvent is not particularly limited and it may be possible to provide N-methyl-2-pyrolidone, γ-butyrolactone, dimethylformamide, dimethylacetamide, tetrahydrofuran, or the like.

A method for synthesizing a diamine represented by general formula (A-1) will be described below.

A method for synthesizing a diamine (aminophenethyl aminophnoxyacetate) represented by a chemical formula of

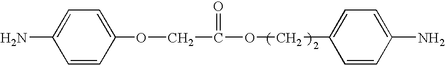

will he described, wherein m is 1, q is 2, and p and r are 0 in general formula (A-1).

First, a dehydration reaction of 4-nitrophenoxyacetic acid represented by a chemical formula of

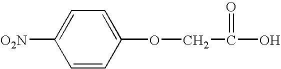

and 2-(4-nitrophenyl)ethanol represented by a chemical formula of

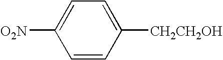

may be caused to obtain a dinitro compound represented by a chemical formula of

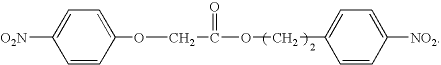

Then, a reduction reaction of the dinitro compound may be caused to obtain aminophenethyl aminophenoxyacetate.

Next, a method for synthesizing a diamine represented by a chemical formula of

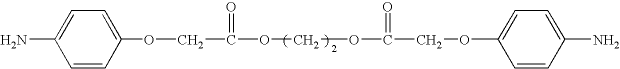

will be described, wherein m, p, q, and r are 1 and n is 2 in general formula (A-1).

First, a dehydration reaction of 4-nitrophenoxyacetic acid and an ethyl ester of 4-nitrophenoxy-2-hydroxyacetic acid represented by a chemical formula of

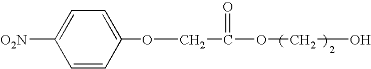

may be caused to obtain a nitro compound represented by a chemical formula of

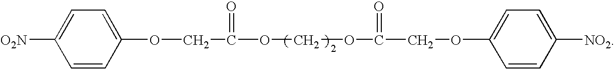

Then, a reduction reaction of the dinitro compound may be caused to obtain a diamine. Similarly, 3-(4-nitrophenoxy)propionic acid and 1,4-butanediol-1-(4-nitrobenzoate) may be used to obtain a diamine wherein m is 2, n is 4, p is 1, and q and r are 0 in general formula (A-1).

Furthermore, similarly, 4-nitrophenoxyacetic acid and 4-nitrophenyl hydroxyacetic acid may be used to obtain a diamine wherein m, n, and p are 1 and q and r are 0.

A polyamic acid according to an embodiment of the present invention may be obtained by causing a ring-opening and addition polymerization reaction of a diamine represented by general formula (A-1) and a diamine including a diamine represented by a general formula of

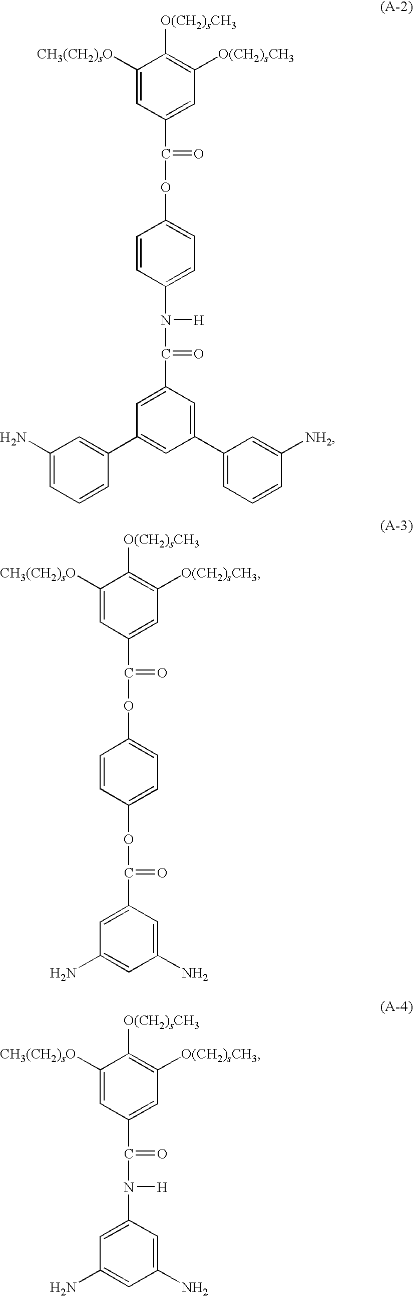

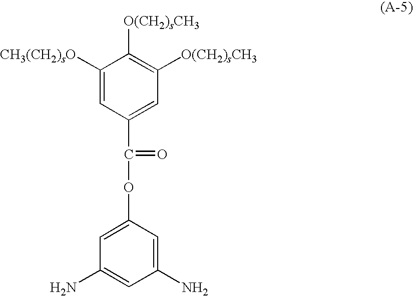

(in which formula, s is an integer of 5-13.), and a tetracarboxylic dianhydride including a tetracarboxylic dianhydride represented by a general formula of

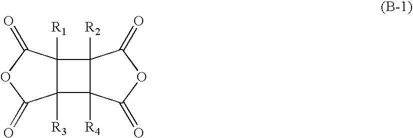

(in which formula, each of R1, R2, R3, and R4 is independently a hydrogen atom, a fluoro group or an alkyl group with a carbon number of 1 or more and 4 or less.) or a tetracarboxylic dianhydride represented by a chemical formula of

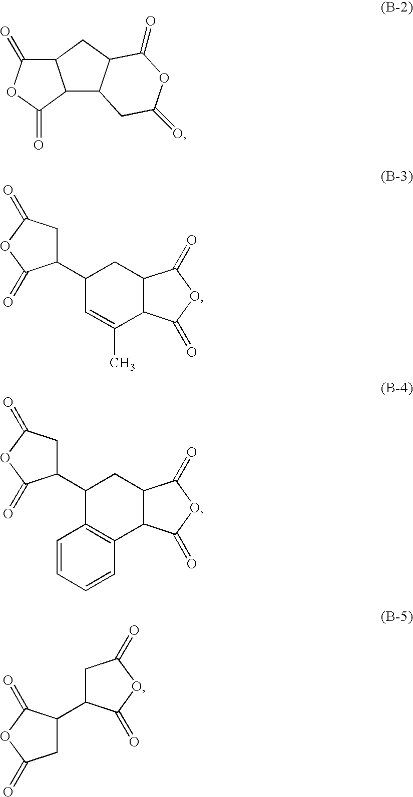

by using a publicly known method. Hence, a polyamic acid according to an embodiment of the present invention may have biodegradability.

Furthermore, a polyimide according to an embodiment of the present invention also includes one obtained by causing a dehydration and ring-closing reaction of a polyamic acid according to an embodiment of the present invention by using a publicly-known method in which a part of its amide bond(s) remains. Hence, biodegradability may be achieved. Moreover, it may be possible to reduce ultraviolet ray irradiation required for changing the surface free energy of a wettability changing layer containing a polyimide according to an embodiment of the present invention.

Herein, if s is less than 5, a change in the surface free energy of a wettability changing layer containing a polyimide according to an embodiment of the present invention which change is caused by ultraviolet ray irradiation may be insufficient, while if it is more than 13, the solubility of a polyimide according to an embodiment of the present invention in an aprotic polar solvent may be insufficient. Furthermore, it may be possible to provide a methyl group, an ethyl group or the like for an alkyl group with a carbon number of 1 to 4 in any of R₁, R₂, R₃, and R₄.

Additionally, diamines for a ring-opening and addition polymerization reaction with a tetracarboxylic dianhydride may include two or more kinds of diamines represented by general formula (A-1) and/or any of diamines represented by general formulas (A-2) to (A-5). Furthermore, tetracarboxylic dianhydrides for a ring-opening and addition polymerization reaction with a diamine may include two or more kinds of tetracarboxylic dianhydrides represented by general formula (B-1) and/or any of tetracarboxylic dianhydrides represented by chemical formulas (B-2) to (B-5).

A wettability changing layer containing a polyimide according to an embodiment of the present invention has an alkyl group(s) derived from a diamine(s) represented by at least one of general formulas (A-2) to (A-5) in its side chain(s), and hence, it may be possible to decrease its surface free energy, that is, to provide a water-repellent property.

Meanwhile, when a wettability changing layer containing a polyimide according to an embodiment of the present invention is irradiated with a ultraviolet ray(s), cleavage of an ester bond or amide bond derived from a diamine(s) represented by at least one of general formulas (A-2) to (A-5) in its side chain may be caused, and hence, it may be possible to increase its surface free energy, that is, to provide a hydrophilic property. Additionally, when cleavage of an ester bond or amide bond in a side chain is caused, a radical is generated and the generated radical may react with water contained in atmosphere immediately to produce a carboxyl group and a hydroxyl group or amino group. Herein, a polyimide according to an embodiment of the present invention also has an ester bond which is derived from a diamine represented by general formula (1) in its main chain but is not conjugated with a benzene ring, and hence, cleavage of the main chain may hardly be caused even if ultraviolet ray irradiation is conducted. Accordingly, it may be possible to maintain the insulating property of a wettability changing layer containing a polyimide according to an embodiment of the present invention even when ultraviolet ray irradiation is conducted.

Moreover, an aromatic diamine other than diamines represented by general formulas (A-1) to (A-5) may be further added to cause a ring-opening and addition polymerization reaction in order to adjust the solubility or film-forming property of a polyimide according to an embodiment of the present invention. Such an aromatic diamine is not particularly limited and it may be possible to provide p-phenylenediamine, 4,4′-methylenedianiline, 4,4′-oxydianiline, bis[3-(4-aminophenoxy)phenyl]sulfone, bis[4-(4-aminophenoxy)phenyl]sulfone, 2,2-bis[4-(4-aminophenoxy)phenyl]propane, 2,2-bis[4-(4-aminophenoxy)phenyl]hexafluoropropane, or the like.

In an embodiment of the present invention, a diamine represented by general formula (1) among diamines for a ring-opening and addition polymerization reaction with a tetracarboxylic dianhydride preferably has a content of 20 to 99 mol % and more preferably 20 to 60 mol %. If this content is less than 20 mol %, the biodegradability of a polyimide according to an embodiment of the present invention or the solubility thereof in an aprotic polar solvent may be insufficient, and if it is more than 99 mol %, a change in the surface free energy of a wettability changing layer containing a polyimide according to an embodiment of the present invention which is caused by ultraviolet ray irradiation may be insufficient.

Furthermore, a wettability changing layer containing a polyimide according to an embodiment of the present invention may be excellent in a resistance thereof to a solvent, because any of tetracarboxylic dianhydrides represented by general formula (B-1) and tetracarboxylic dianhydrides represented by chemical formulas (B-2) to (B-5) has a hydrophobic group. Moreover, a wettability changing layer containing a polyimide according to an embodiment of the present invention may be excellent in an insulating property thereof, because any of tetracarboxylic dianhydrides represented by general formula (B-1) and chemical formulas (B-2) to (B-5) has a carbonyl group which is not conjugated with a benzene ring.

A polyamic acid according to an embodiment of the present invention is preferably soluble in an aprotic polar solvent. Accordingly, it may be possible to apply a solution of a polyamic acid according to an embodiment of the present invention to a substrate and subsequently cause a dehydration and ring-closing reaction to form a wettability changing layer. Herein, a solution of a polyamic acid according to an embodiment of the present invention may further contain a polyimide according to an embodiment of the present invention. Furthermore, two or more kinds of polyamic acids according to an embodiment of the present invention and/or polyimides according to an embodiment of the present invention may be used in combination.

Such an aprotic polar solvent is not particularly limited and it may be possible to provide N-methyl-2-pyrolidone, γ-butyrolactone, dimethylformamide, dimethylacetamide, tetrahydrofuran, or the like.

A method for applying a solution of a polyamic acid according to an embodiment of the present invention to a substrate is not particularly limited and it may be possible to provide a dip coating method, a spin coating method, a decalcomania method, a roll coating method, an ink jet method, a spraying method, a brush coating method, or the like.

A polyamic acid according to an embodiment of the present invention preferably has a number-average molecular weight of 3×10³ to 5×10⁵. Accordingly, it may be possible for a polyimide according to an embodiment of the present invention which is obtainable by means of a dehydration and ring-closing reaction of a polyamic acid according to an embodiment of the present invention to have a glass transition point of 200 to 400° C. Herein, such a number average molecular weight is a molecular weight of a polystyrene standard which is measured by using GPC (gel permeation chromatography).

The reaction rate of a dehydration and ring-closing reaction of a polyamic acid is preferably 90 to 100% and more preferably 95 to 100%. If the reaction rate is less than 90%, when a wettability changing layer containing a polyimide according to an embodiment of the present invention is used as a gate-insulating film of an organic thin-film transistor, it may be impossible to form a good interface between the wettability changing layer and an organic semiconductor layer. As a result, the variation of a threshold voltage of an organic thin-film transistor may be large. Herein, it may be possible to measure the reaction rate of a dehydration and ring-closing reaction of a polyamic acid while a polyimide in dimethyl sulfoxide (CMSO)-d₆ is dissolved to measure ¹H-NMR thereof and the rate of a remaining amide bond(s) is calculated from the ratio of a peak surface area thereof.

A polyimide according to an embodiment of the present invention is preferably soluble in an aprotic polar solvent. Accordingly, it may be possible to apply to a substrate a solution in which a polyimide according to an embodiment of the present invention is dissolved in an aprotic polar solvent, to form a wettability changing layer. Herein, two or more kinds of polyimides according to an embodiment of the present invention may be used in combination.

Such an aprotic polar solvent is not particularly limited and it may be possible to provide N-methyl-2-pyrolidone, γ-butyrolactone, dimethylformamide, dimethylacetamide, tetrahydrofuran, or the like.

A method for applying a solution of a polyimide according to an embodiment of the present invention to a substrate is not particularly limited and it may be possible to provide a dip coating method, a spin coating method, a decalcomania method, a roll coating method, an ink jet method, a spraying method, a brush coating method, or the like.

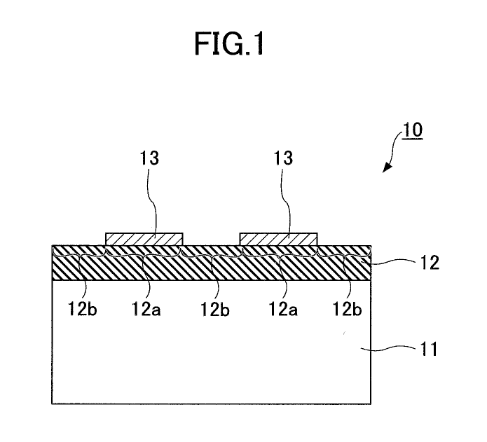

FIG. 1 illustrates one example of a laminated structure according to an embodiment of the present invention. In a laminated structure 10, a wettability changing layer 12 containing a polyimide according to an embodiment of the present invention is formed on a substrate 11. Herein, the wettability changing layer 12 is composed of ultraviolet-ray-irradiated areas 12a which have been irradiated with ultraviolet rays to increase surface free energies thereof and ultraviolet-ray-non-irradiated areas 12b which have not been irradiated with an ultraviolet ray. Additionally, the ultraviolet-ray-non-irradiated areas 12b which have a width of 1 to 5 μm are formed between the ultraviolet-ray-irradiated areas 12a. Furthermore, electrically conductive layers 13 are formed on the ultraviolet-ray-irradiated areas 12a of the wettability changing layer 12 to provide a laminated structure. Thus, it may be possible to readily form electrically conductive layers 13 having fine patterns.

The material for making the substrate 11 is not particularly limited and it may be possible to provide a glass; a resin such as a polyester, a polycarbonate, a polyacrylate, a polyether sulfone, a polyethylene terephthalate, or a polyethylene naphthalate; a metal such as SUS; or the like, wherein a resin may be preferable in the case where a flexibility is desired.

Additionally, when the film-forming property of a polyamic acid according to an embodiment of the present invention or a polyimide according to an embodiment of the present invention is insufficient, a material excellent in a film-forming property may be added into a solution of the polyamic acid according to an embodiment of the present invention or the polyimide according to an embodiment of the present invention.

The thickness of the wettability changing layer 12 is usually 30 nm to 3 μm and preferably 50 nm to 1 μm. If the thickness of the wettability changing layer 12 is less than 30 nm, it may be difficult to conduct uniform formation thereof, and if it is more than 3 μm, the shape of the surface thereof may be deteriorated.

Furthermore, it may be possible to apply and subsequently heat or conduct ultraviolet ray irradiation to, a coating fluid containing an electrically conductive material to form the electrically conductive layers 13.

Such an electrically conductive material is not particularly limited and it may be possible to provide a metal such as gold, silver, copper, aluminum, or calcium; a carbonic material such as a carbon black, a fullerene, or a carbon nanotube; an organic π-conjugate polymer such as a polythiophene, a polyaniline, a polypyrrole, a polyfluorene, or a derivative thereof; or the like, wherein two or more kinds thereof may be used in combination. Furthermore, different electrically conductive materials may be used for forming a gate electrode and source and drain electrodes.

A coating fluid containing an electrically conductive material is not particularly limited and it may be possible to provide a solution in which an electrically conductive material is dissolved in a solvent, a solution in which a precursor of an electrically conductive material is dissolved in a solvent, a dispersion fluid in which an electrically conductive material is dispersed in a dispersion medium, a dispersion fluid in which a precursor of an electrically conductive material is dispersed in a dispersion medium, or the like.

Such a solvent or dispersion medium is not particularly limited, and it may be possible to provide water, each kind of an alcohol, or the like, because the wettability changing layer 12 may be provided without causing a significant damage. Furthermore, a solvent or dispersion medium such as N,N-dimethylformamide, N,N-dimethylacetamide, 2-pyrolidone, N-methyl-2-pyrolidone, n-ethyl-2-pyrolidone, N-vinyl-2-pyrolidone, N-methylcaprolactam, dimethyl sulfoxide, or tetramethylurea may also be used as long as the wettability changing layer 12 is provided without causing a significant damage.

For a coating fluid containing an electrically conductive material, it may be possible to provide a dispersion fluid in which a particle(s) of a metal such as silver, gold, nickel, or copper is/are dispersed in an organic dispersion medium or solvent or water, an aqueous solution of a doped PANI (polyaniline) or an electrically conductive polymer in which PEDOT (polyethylenedioxythiophene) is doped with PSS (polystyrenesulfonic acid), or the like.

A method for applying a coating fluid containing an electrically conductive material is not particularly limited and it may be possible to provide a spin coating method, a dip coating method, a screen printing method, an offset printing method, an ink jet method, or the like. Among these, an ink jet method capable of ejecting a small liquid drop is preferable, because it may be susceptible to the surface free energy of the wettability changing layer 12. When a head at the normal level to be used in a printer is used, the resolution and positioning precision of an ink jet method are 30 μm and ±15 μm, respectively, but it may be possible to form electrically conductive layers 13 having a fine pattern when the difference between the surface free energies of the wettability changing layer 12 is utilized.

It may be possible to apply a laminated structure according to an embodiment of the present invention to a gate electrode of an organic thin-film transistor and an interconnection thereof, source and drain electrodes of an organic thin-film transistor and an interconnection thereof, or the like.

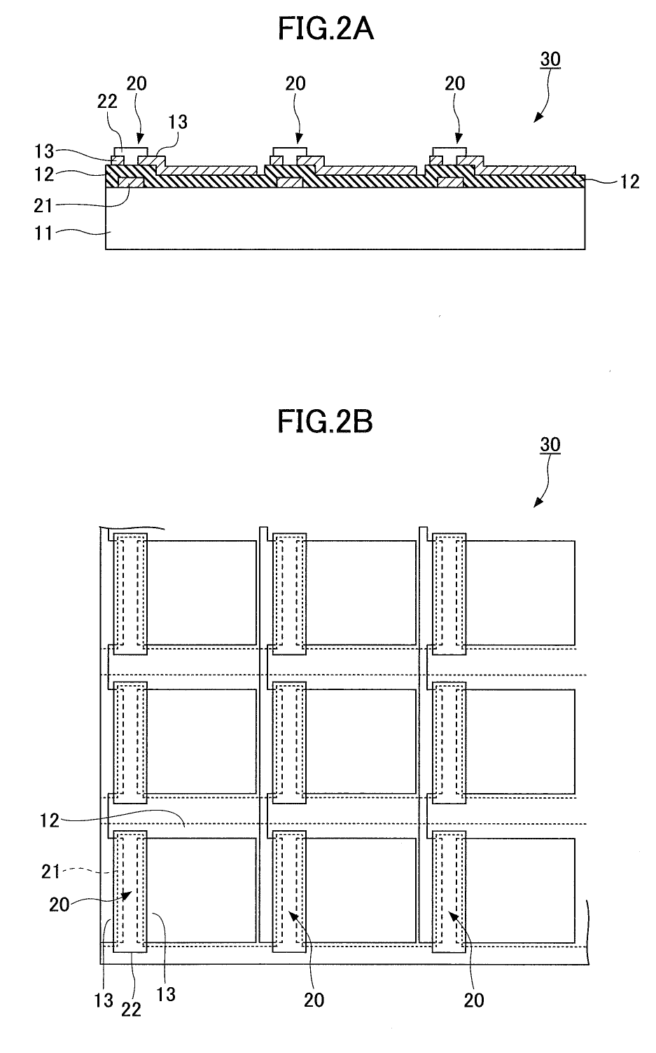

FIGS. 2A and 2B illustrate a thin-film transistor array as an example of an electronic element array according to an embodiment of the present invention. A thin-film transistor array 30 has a plurality of bottom-gate-type thin-film transistors 20. Herein, FIGS. 2A and 2B are a cross-sectional view and top view thereof, respectively. Additionally, in FIGS. 2A and 2B, the same reference numeral is provided to the same configuration as that of FIG. 1 and the description thereof is omitted.

In a thin-film transistor 20, gate electrodes 21 are formed on a substrate 11. Furthermore, a wettability changing layer 12 as a gate-insulating film is formed on the substrate 11 on which the gate electrodes 21 are formed, and electrically conductive layers 13 as source and drain electrodes are formed on ultraviolet-ray-irradiated areas of the wettability changing layer 12. Moreover, semiconductor layers 22 are formed on channel areas between the source and drain electrodes. Thus, it may he possible to readily form gate electrodes or source and drain electrodes having a fine pattern.

Each of the semiconductor layers 22 may be either an inorganic semiconductor layer or an organic semiconductor layer, and an organic semiconductor layer is preferable, because it may be possible to simplify, or reduce the cost of, a process for producing a thin-film transistor.

A material for making an inorganic semiconductor layer is not particularly limited and it may be possible to provide CdSe, CdTe, Si, or the like.

A method for forming an inorganic semiconductor layer is not particularly limited and it may be possible to provide a method using a vacuum process such as sputtering, a sol-gel method, or the like.

A material for making an organic semiconductor layer is not particularly limited and it may be possible to provide an organic low molecule such as pentacene, anthracene, tetracene, or phthalocyanine; a polyacetylene-type electrically conductive polymer; a polyphenylene-type electrically conductive polymer such as a poly(p-phenylene) or a derivative thereof or a polyphenylenevinylene or a derivative thereof; a heterocyclic electrically conductive polymer such as a polypyrrole or a derivative thereof, a polythiophene or a derivative thereof, or a polyfuran or a derivative thereof; an ionic electrically conductive polymer such as a polyaniline or a derivative thereof; or the like.

A method for forming an organic semiconductor layer is not particularly limited and it may be possible to provide a spin coating method, a spray coating method, a printing method, an ink jet method, or the like.

Additionally, instead of forming the gate electrodes 21, a wettability changing layer 12 may be formed on the substrate 11 and electrically conductive layers 13 as gate electrodes may be formed on ultraviolet-ray-irradiated areas of the wettability changing layer 12.

Herein, two polyimides according to an embodiment of the present invention which is contained in the wettability changing layers 12 may be identical to or different from each other. Furthermore, two electrically conductive materials contained in the electrically conductive layers 13 may be identical to or different from each other.

Moreover, when the volume resistivity of the wettability changing layer 12 is small, an insulator layer with a volume resistivity larger than that of the wettability changing layer 12 and the wettability changing layer 12 may be laminated in order to form a gate-insulating film. When such as gate-insulating film is irradiated with an ultraviolet ray(s), it may be possible for the wettability changing layer 12 to absorb an ultraviolet ray(s) and accordingly to suppress degradation of the insulating property of the insulator layer.

A material for making an insulator layer is not particularly limited and it may be possible to provide a polyimide, a polyamide-imide, an epoxy resin, a silsesquioxane, a polyvinylphenol, a polycarbonate, a fluororesin, a poly(p-xylylene), or the like.

A method for forming an insulator layer is not particularly limited and it may be possible to provide a decalcomania method, a spin coating method, a dip coating method, or the like.

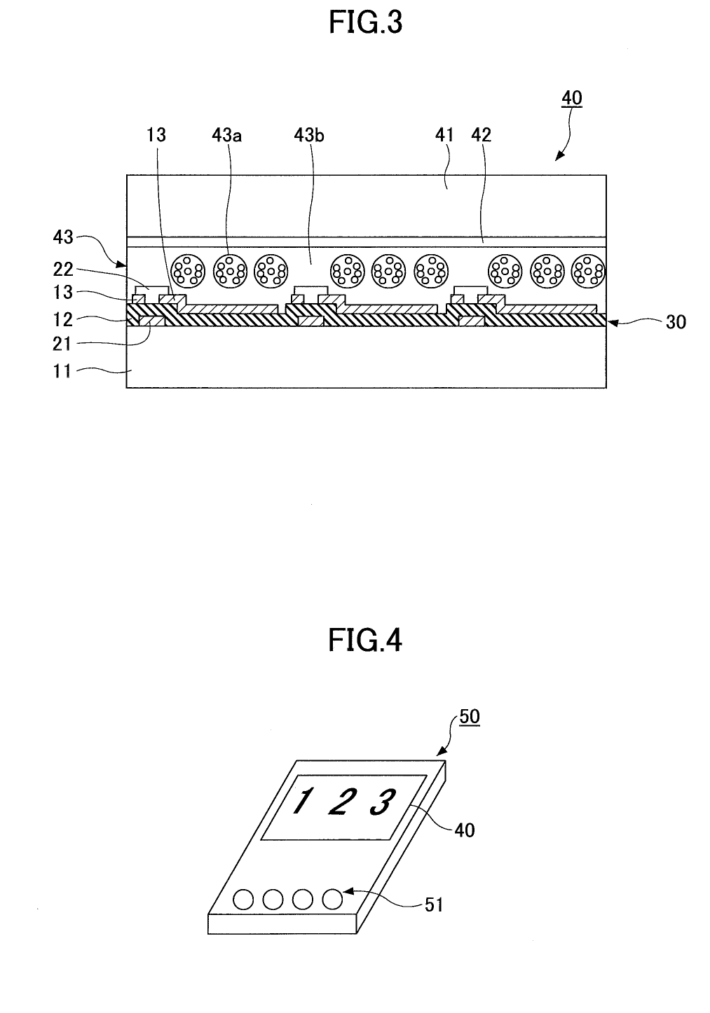

FIG. 3 illustrates an electrophoretic panel as an example of an image display medium according to an embodiment of the present invention. In an electrophoretic panel 40, a transparent electrode 42 is formed on a transparent substrate 41 and an image display layer 43 composed of microcapsules 43a as electrophoretic elements and a binder 43b is formed on the electrode 42. Herein, the microcapsules 43a contain, for example, a white titanium oxide particle(s) and Isopar L (produced by Exxon Mobil Corporation) colored with oil blue therein. Furthermore, the image display layer 43 is joined with a thin-film transistor array 30 as an active matrix substrate.

Additionally, an image display medium according to an embodiment of the present invention is not particularly limited to an electrophoretic panel and may be a liquid crystal panel or organic EL panel in which an active matrix substrate combined with an image display element such as a liquid crystal element or organic EL element, or the like. Furthermore, it may be possible to use an image display medium according to an embodiment of the present invention as an electronic paper.

FIG. 4 illustrates a pocket PC as an example of an image display device according to an embodiment of the present invention. A pocket PC 50 has an electrophoretic panel 40 as a flat screen and an image is displayed by inputting image information from an input part 51.

In addition, it may also be possible to apply an image display medium according to an embodiment of the present invention to a copying machine, and it may also be possible to embed it in a sheet part or front glass surface of a traffic movement medium such as an automobile or an aircraft.

Furthermore, it may be possible to apply an electronic element array according to an embodiment of the present invention to a solar cell, an RFID tag, or the like, other than image display media.

Although a polyamic acid and polyimide capable of being applied to a laminated structure according to an embodiment of the present invention have been described above, a polyamic acid according to an embodiment of the present invention is not limited to such a polyamic acid as described above and it is only necessary to be a polyamic acid obtainable by means of a ring-opening and addition polymerization reaction of a diamine(s) including a diamine represented by general formula (A-1) with a tetracarboxylic dianhydride. Furthermore, it is only necessary for a polyimide according to an embodiment of the present invention to be a polyimide obtainable by means of a dehydration and ring-closing reaction of a polyamic acid that is thus obtainable.

Herein, a diamine other than diamines represented by general formula (A-1) is not particularly limited as long as it is possible to cause a ring-opening and addition polymerization reaction with a tetracarboxylic dianhydride, and it may be possible to provide 4,4′-diaminodiphenyl ether, 4,4′-diaminodiphenylmethane, 4,4′-diaminobenzophenone, or the like, wherein two or more kinds thereof may be used in combination.

Furthermore, a tetracarboxylic dianhydride is not particularly limited as long as it is possible to cause a ring-opening and addition polymerization reaction with a diamine, and it may be possible to provide benzophenone-3,4,3′,4′-tetracarboxylic dianhydride, diphenylsulfone-3,4,3′,4′-tetracarboxylic dianhydride, 1-carboxymethyl-2,3,5-cyclopentane tricarboxylic-2,6:3,5-dianhydride, or the like, wherein two or more kinds thereof may be used in combination.

### Practical Examples

[Synthesis of Diamine]

After 0.8 g (4.12 mmol) of 4-nitrophenoxyacetic acid, 2.04 g (12.2 mmol) of 2-(4-nitrophenyl)ethanol, and 0.40 g of 4-dimethylaminopyridine as a basic catalyst were mixed, 6.5 ml of tetrahydrofuran was added to cause dissolution thereof. Then, after cooling to 0° C. was conducted and 0.92 g of dicyclohexylcarbodiimide as a dehydration catalyst was added, agitation was conducted at 50° C. for 24 hours. Furthermore, after a produced dicyclohexylurea was filtered out by means of filtration under a reduced pressure, tetrahydrofuran was distilled out by using an evaporator. After an obtained solid component was dissolved in dichloromethane so that its content was 5% by mass, dichloromethane was distilled out by using an evaporator to obtain a dinitro compound.

After the obtained dinitro compound, 20 ml of ethanol, and 3 ml of tetrahydrofuran were mixed, 0.104 g of palladium-carbon as a catalyst was added to cause catalytic hydrogenation reaction at 0.3 MPa. Then, after filtration was conducted by using a double filter paper to remove palladium-carbon, tetrahydrofuran and ethanol were distilled out by using an evaporator. Furthermore, after filtration was conducted under a reduced pressure, vacuum drying was conducted at room temperature to obtain aminophenethyl aminophenoxyacetate.

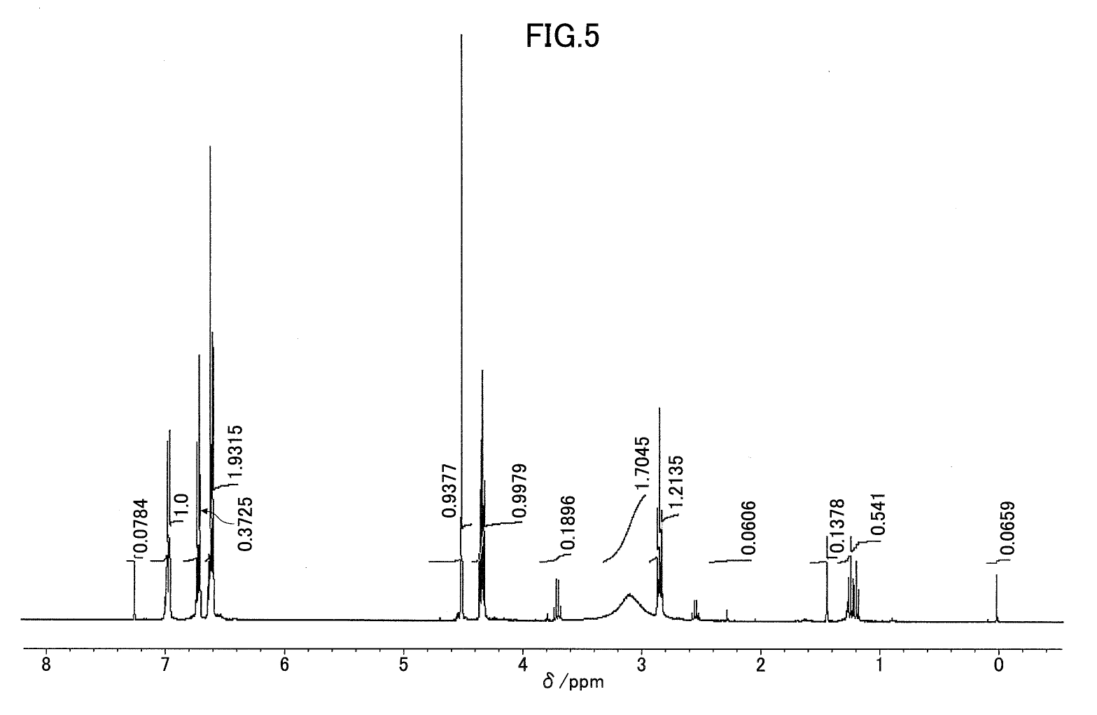

FIG. 5 illustrates a ¹H-NMR spectrum of the obtained aminophenethyl aminophenoxyacetate.

### Practical Example 1

N-methyl-2-pyrolidone was added into a mixture of the aminophenethyl aminophenoxyacetate, a diamine represented by a chemical formula of:

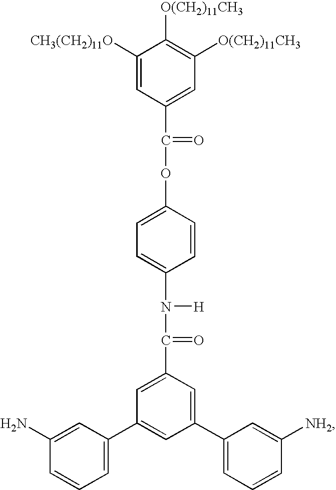

and a tetracarboxylic dianhydride represented by a chemical formula of:

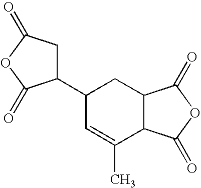

(molar ratio 1:1:2) so that the concentration of its solid component was 20% by mass, and a ring-opening and addition polymerization reaction was conducted under argon atmosphere at room temperature for 24 hours to obtain a polyamic acid represented by a chemical formula of:

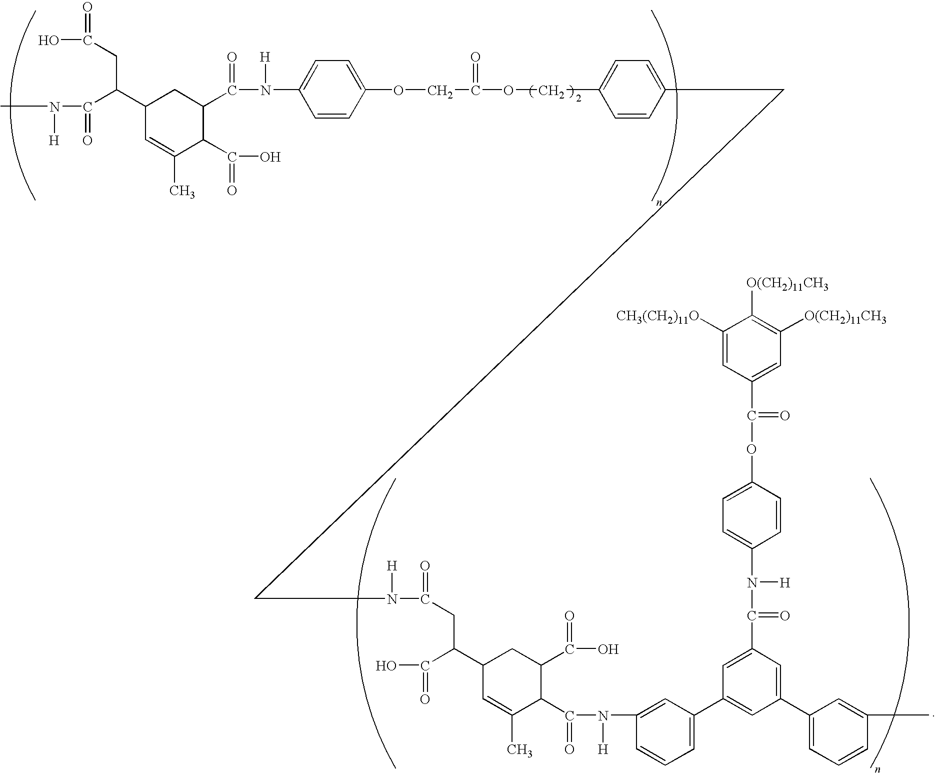

The number average molecular weight of the obtained polyamic acid was measured by using a GPC, and as a result, was 2×10⁴.

Then, N-methyl-2-pyrolidone was further added so that the concentration of a solid component of the polyamic acid was 10% by mass, and 5 equivalents of pyridine and 4 equivalents of acetic anhydride based on the total content of the diamine and the tetracarboxylic dianhydride were added to cause a dehydration and ring-closing reaction under argon atmosphere at 120° C. for 4 hours. After the obtained reaction liquid was added into stirred methanol to cause precipitation, filtration was conducted under a reduced pressure. Furthermore, after washing was conducted with methanol, vacuum drying was conducted at 80° C. to obtain a polyimide represented by a chemical formula of:

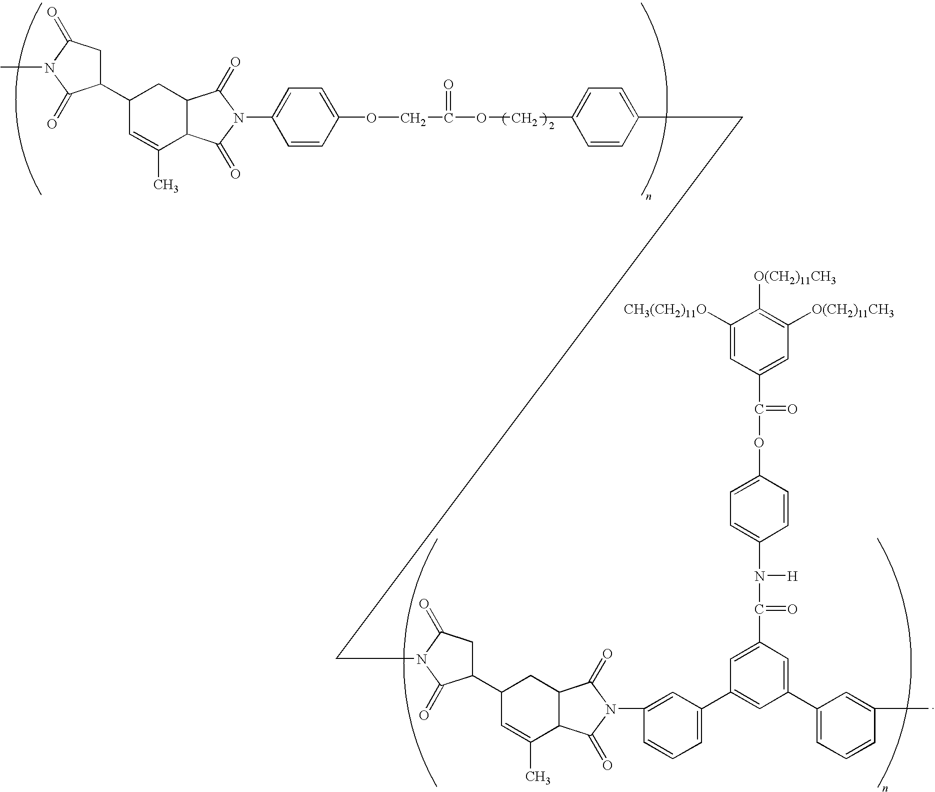

### Practical Example 2

N-methyl-2-pyrolidone was added into a mixture of the aminophenethyl aminophenoxyacetate and a tetracarboxylic dianhydride represented by a chemical formula of:

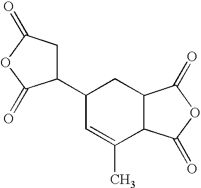

(molar ratio 1:1) so that the concentration of its solid component was 20% by mass, and a ring-opening and addition polymerization reaction was caused at room temperature for 24 hours to obtain a polyamic acid represented by a chemical formula of:

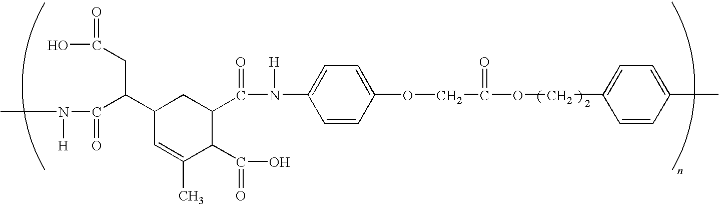

The number average molecular weight of the obtained polyamic acid was measured by a GPC, and as a result, was 2×10⁵.

Then, N-methyl-2-pyrolidone was further added so that the concentration of a solid component of the polyamic acid was 10% by mass, and 5 equivalents of pyridine and 4 equivalents of acetic anhydride based on the total content of the diamine and the tetracarboxylic dianhydride were added to cause a dehydration and ring-closing reaction under argon atmosphere at 120° C. for 4 hours. After the obtained reaction liquid was added into stirred methanol to cause precipitation, filtration was conducted under a reduced pressure. Furthermore, after washing was conducted with methanol, vacuum drying was conducted at 80° C. to obtain a polyimide represented by a chemical formula of:

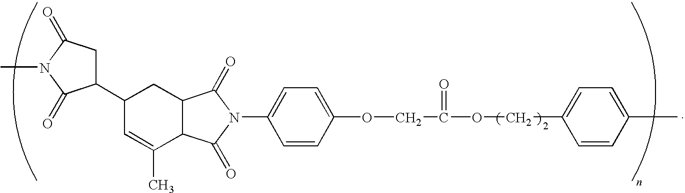

### Comparative Example 1

A polyimide represented by a chemical formula of:

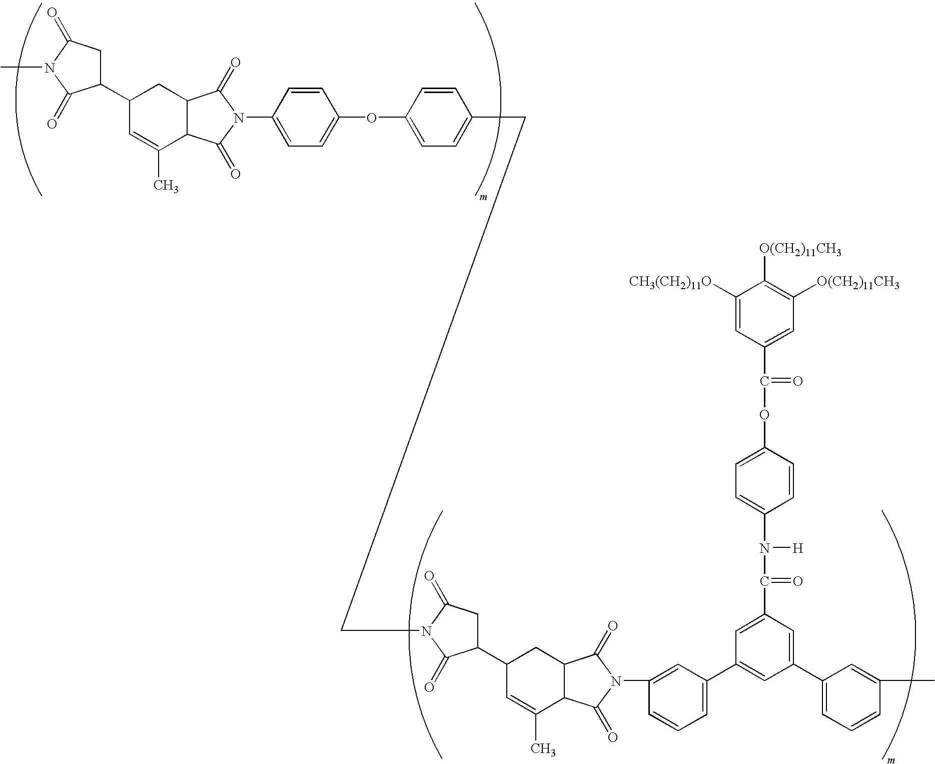

was obtained similarly to practical example 1, except that a diamine represented by a chemical formula of:

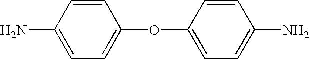

was used instead of the aminophenethyl aminophenoxyacetate. Herein, the number average molecular weight of the polyamic acid was 1.2×10⁴.

### Comparative Example 2

A polyimide represented by a chemical formula of:

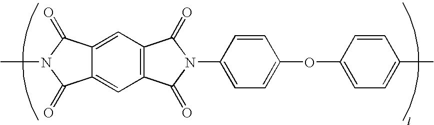

was obtained similarly to practical example 2 except that a diamine represented by a chemical formula of:

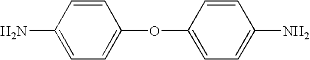

was used as a diamine and pyromellitic dianhydride was used as a tetracarboxylic dianhydride. Herein, the number average molecular weight of the polyamic acid was 5.4×10⁴.

[Biodegradability]

A solution in which any one of the polyimides in the practical example and comparative examples was dissolved in N-methyl-2-pyrolidone was applied onto a glass substrate by using a spin coating method and subsequently heated by using a hot plate to obtain a square-shaped film having sides of 1.5 cm.

The films in the practical example and comparative examples were left in 4 kg of compost (originating from pruned branches, cattle dung, and weeds).

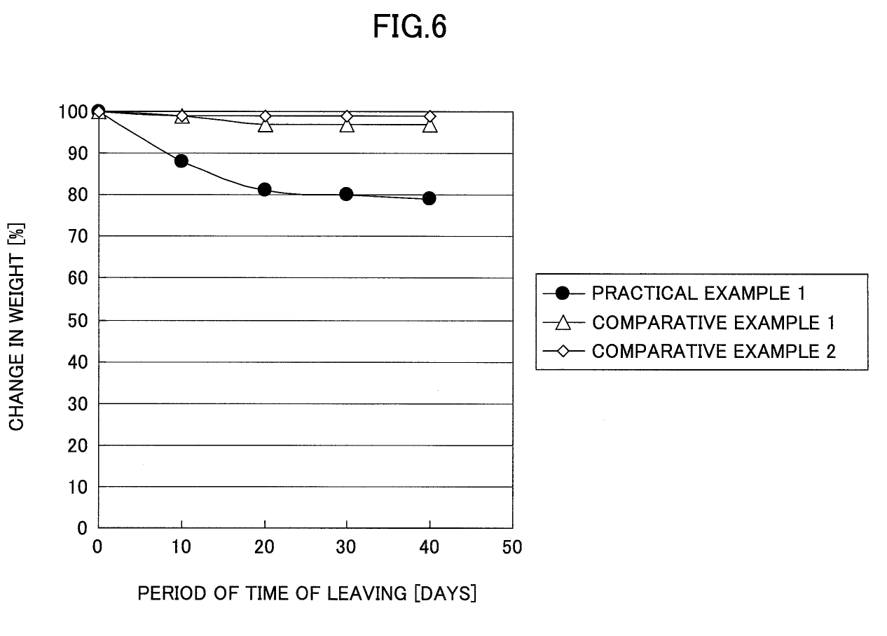

FIG. 6 illustrates changes in the weights of the films in the practical example and comparative examples versus a period of time of leaving in the compost. From FIG. 6, it is seen that the film in practical example 1 had biodegradability and accordingly the weight thereof decreased whereas the weights of the films in the comparative examples hardly decreased.

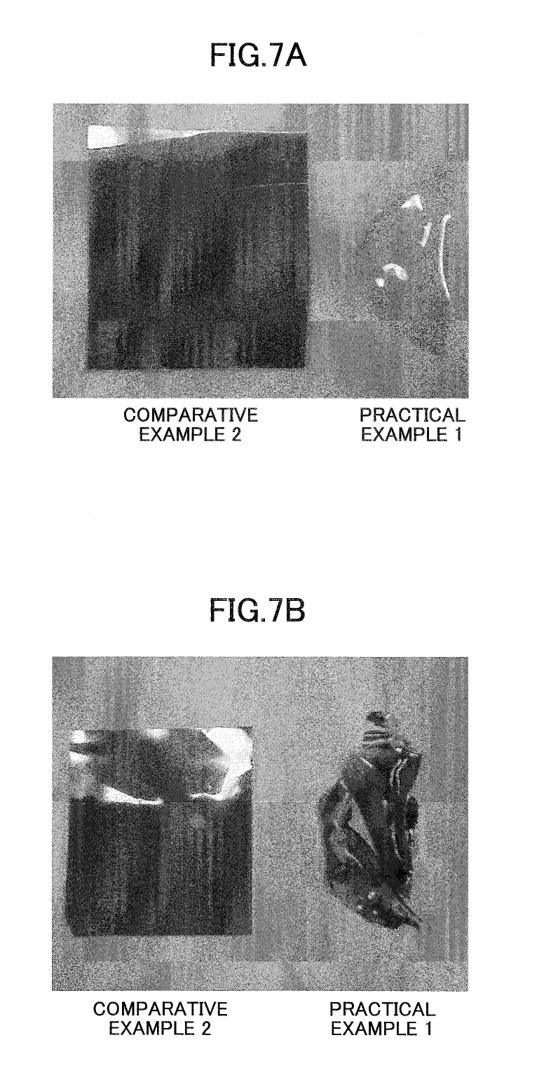

FIGS. 7A and 7B illustrate the films in practical example 1 and comparative example 2 before leaving in the compost and after leaving for 30 days in the compost, respectively. From FIGS. 7A and 7B, it is seen that the film in practical example 1 had biodegradability and accordingly the shape thereof was changed whereas the shape of the film in comparative example 2 was hardly changed.

Then, the film in practical example 1 was left in 4 kg of compost (originating from pruned branches, cattle dung, and weeds) in an aerobic condition at 55° C. and the number average molecular weights of the film after 20 days and 45 days were measured by using a GPC. As a result, whereas the number average molecular weight decreased when leaving in the compost, the number average molecular weight was hardly changed when leaving in the aerobic condition at 55° C.

[Contact Angle]

A solution in which any one of the polyimides in the practical example and comparative examples was dissolved in N-methyl-2-pyrolidone was applied onto a substrate 11 made of glass by using a spin coating method and subsequently heated by using a hot plate to form a wettability changing layer 12.

Then, after the wettability changing layer 12 was irradiated with a predetermined amount (see Table 1) of ultraviolet rays by using a high pressure mercury lamp, the contact angle of a dispersion liquid in which silver nano-particles were dispersed in an aqueous dispersion medium (referred to as a silver nano-ink, below) was measured by using a liquid drop method. The results of the measurement are presented in Table 1.

From Table 1, it is seen that the wettability changing layer 12 in any of the practical example 1 and comparative example 1 contained a polyimide having an alkyl group originating from a diamine in a side chain thereof, and accordingly, the surface thereof was water-repellent so that the contact angle of the silver nano-ink was 30° or more. On the other hand, the wettability changing layer 12 in comparative example 2 contained a polyimide having no alkyl group originating from a diamine in a side chain thereof, and accordingly, the surface thereof was hydrophilic so that the silver nano-ink generally spread wettably. Meanwhile, when the wettability changing layer 12 in any of practical example 1 and comparative example 1 was irradiated with ultraviolet rays, it was considered that an ester bond or amide bond originating from a diamine which was possessed in a side chain of the polyimide contained in the wettability changing layer 12 was cleaved, and accordingly, the surface thereof was hydrophilic so that the silver nano-ink spread wettably.

[Patterning Characteristic]

A solution in which the polyimide in any one of the practical example and comparative examples was dissolved in N-methyl-2-pyrolidone by using a spin coating method was applied onto a substrate 11 made of glass and subsequently heated by using a hot plate to form a wettability changing layer 12.

Then, the wettability changing layer 12 was irradiated with a predetermined amount (see Table 1) of ultraviolet rays through a line-shaped photo-mask with an interval of 5 μm by using a high-pressure mercury lamp. Furthermore, the silver nano-ink was applied onto ultraviolet-ray-irradiated areas by using an ink jet method and subsequently baked at 200° C. by using an oven so that electrically conductive layers 13 were formed to obtain a laminated structure 10 (see FIG. 1).

Then, the electrically conductive layers 13 were observed by using a metallographic microscope to evaluate the patterning characteristic thereof. The results of such evaluation are presented in Table 1. Herein, criteria were provided such that A indicated that all the line-shaped electrically conductive layers 13 with an interval of 5 μm were formed, B indicated that almost all of them were formed, and C indicated that none of them was formed.

From Table 1, it is seen that the results of evaluation of the patterning characteristic correlated with the results of evaluation of the change in the contact angle. That is, the difference between the surface free energies of ultraviolet-ray-irradiated areas 12a and ultraviolet-ray-non-irradiated areas 12b of the wettability changing layer 12 in any of practical example 1 and comparative example 1 was large, and hence, the line-shaped electrically conductive layers 13 with an interval of 5 μm were formed even if the amount of ultraviolet ray irradiation was 15 J/cm² or less. On the other hand, the difference between the surface free energies of ultraviolet-ray-irradiated areas 12a and ultraviolet-ray-non-irradiated areas 12b of the wettability changing layer 12 in comparative example 2 was insufficient, and hence, no line-shaped electrically conductive layers 13 with an interval of 5 μm was formed when the amount of ultraviolet ray irradiation was 15 J/cm² or less.

TABLE 1

| | | Amount of ultraviolet ray irradiation | | | | |
|---|---|---|---|---|---|---|
| | | [J/cm²] | | | | |
| | | 0 | 2 | 5 | 10 | 15 |
| Practical | Contact angle | 34 | 14 | 4 | 4 | 4 |
| example 1 | [°] | | | | | |
| | Patterning | C | B | A | A | A |
| | property | | | | | |
| Comparative | Contact angle | 33 | 13 | 5 | 4 | 4 |
| example 1 | [°] | | | | | |
| | Patterning | C | B | A | A | A |
| | property | | | | | |
| Comparative | Contact angle | 10 | 10 | 10 | 9 | 9 |
| example 2 | [°] | | | | | |
| | Patterning | C | C | C | C | C |
| | property | | | | | |

[Manufacturing of a Thin-Film Transistor Array]

Aluminum was deposited onto a substrate 11 made of polyethylene naphthalate in vacuum by using a metal mask to form gate electrodes 21 with a thickness of 50 nm. Then, a solution in which a mixture of the polyimide in practical example 1 or comparative example 1 or 2 and the polyimide in practical example 2 with a weight ration of 1:2 was dissolved in N-methyl-2-pyrolidone was applied onto the substrate 11 on which the gate electrodes 21 were formed, by using a spin coating method, and baked at 170° C. to form a wettability changing layer 12 with a thickness of 600 nm (as a gate insulating film). Furthermore, the gate insulating film was irradiated with ultraviolet rays at 10 J/cm² through a photo-mask by using a high-pressure mercury lamp. Then, the silver nano-ink was applied onto the ultraviolet-ray-irradiated areas by using an ink jet method and subsequently baked at 170° C. by using an oven to form electrically conductive layers 13 (as source electrodes and drain electrodes with a channel length of 5 μm). Furthermore, a solution of a compound represented by a chemical formula of:

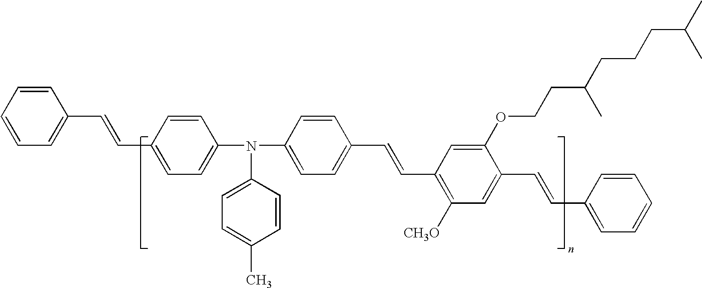

in xylene was applied onto channel areas between the source and drain electrodes by using an ink jet method and subsequently was heated at 120° C. by using an oven whereby semiconductor layers 22 with a thickness of 30 nm were formed to obtain a thin-film transistor array 30 (see FIGS. 2A and 2B).

Table 2 presents the results of evaluation of the transistor characteristics of the obtained thin-film transistor array 30.

TABLE 2

| | | |
|---|---|---|
| | Field-effect | |
| | mobility | On-off |
| Polyimide | [cm²/V · second] | ratio |

| | | | |
|---|---|---|---|
| Practical | Practical | 5 × 10⁻³ | 5 digits |
| example 1 | example 2 | | |
| Comparative | Practical | 4 × 10⁻³ | 5 digits |
| example 1 | example 2 | | |

From Table 2, the thin-film transistor array 30 manufactured by using the polyimide in practical example 1 or comparative example 1 did not only have a large field-effect mobility but also a small gate leak electric current and a large on-off ratio. It is considered that this is because the insulating property of the constitutional units originating from the tetracarboxylic dianhydride was high and, in addition, the surface smoothness of the wettability changing layer 12 was high. Furthermore, the field-effect mobility of the thin-film transistor array 30 in the case where the polyimide in practical example 1 was used was larger than that in the case where the polyimide in comparative example 1 was used. It is considered that this is because the polyimide in practical example 1 had a higher compatibility with the polyimide in practical example 2 and a higher surface smoothness of the wettability changing layer 12, as compared to the polyimide in comparative example 1. On the other hand, a thin-film transistor array 30 manufactured by using the polyimide in comparative example 2 exhibited no transistor characteristic while a wettability changing layer 12 thereof was whitely turbid. It is considered that this is because the surface smoothness of the wettability changing layer 12 was low and, in addition, the insulating property of the constitutional units originating from the tetracarboxylic dihydride was low.

[Manufacturing of an Electrophoretic Panel]

An electrophoretic panel 40 (see FIG. 3) was manufactured by using a thin-film transistor array 30 which was manufactured by using the polyimide in practical example 1. Specifically, a coating liquid in which microcapsules 43a containing titanium oxide particles and Isopar colored with oil blue therein and an aqueous solution of polyvinyl alcohol 43b were mixed was applied onto a transparent electrode 42 made of ITO which was formed on a transparent substrate 41 made of polycarbonate, to form an image display layer 43 composed of microcapsules 43a and polyvinyl alcohol 43b. Furthermore, the thin-film transistor array 30 was bonded to the image display layer 43 such that the substrate 11 and the transparent substrate 41 were the outermost faces, to obtain an electrophoretic panel 40.

While a driver IC for a scanning signal was connected to a bus line connecting to the gate electrode 21 of the electrophoretic panel 40 and a driver IC for a data signal was connected to a bus line connecting to the source electrode, switching of an image was conducted every 0.5 seconds, whereby it was possible to display a good static image.

[Appendix]

One object of at least one embodiment of the present invention may be to provide a diamine having biodegradability and being capable of causing a ring-opening and addition polymerization reaction with a tetracarboxylic dianhydride in good yield, a polyamic acid to be obtained by causing a ring-opening and addition polymerization reaction of the diamine with a tetracarboxylic dianhydride, and a polyimide to be obtained by causing a dehydration and ring-closing reaction of the polyamic acid.

Also, another object of at least one embodiment of the present invention may be to provide a laminated structure in which a wettability changing layer is formed which has biodegradability and allows for a reduction in ultraviolet ray irradiation for changing its surface free energy, and an electronic element array, image display medium and image display device having the laminated structure.

Embodiment (1) of the present invention is a diamine characterized by being represented by a general formula of

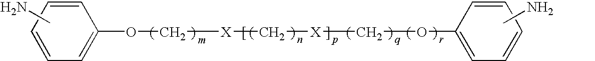

(in which formula, X is an ester bond, each of in and n is independently a natural number, each of p and r is independently 0 or 1, q is an integer of 0 or more, and a sum of m, n, and q is 20 or less. Herein, when q is 0, p is 1 and r is 0.).

Embodiment (2) of the present invention is a polyamic acid characterized by being to be obtained by causing a ring-opening and addition polymerization reaction of a diamine including the diamine as described in embodiment (1) above and a tetracarboxylic dianhydride.

Embodiment (3) of the present invention is a polyamic acid to be obtained by causing a ring-opening and addition polymerization reaction of diamines, including a first diamine and a second diamine, and a tetracarboxylic dianhydride, characterized in that the first diamine is the diamine as described in embodiment (1) above, the second diamine is a diamine represented by a general formula of

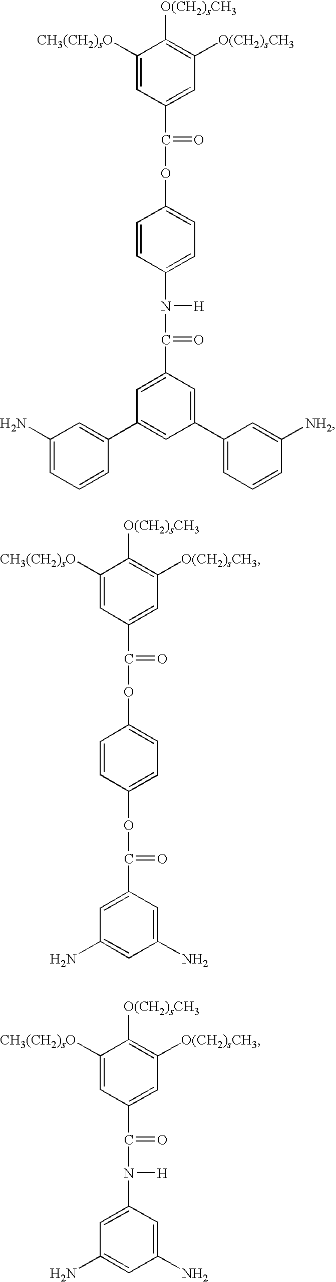

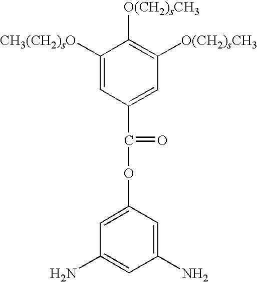

(in which formula, s is an integer of 5 or more and 13 or less.), and the tetracarboxylic dianhydride includes a tetracarboxylic dianhydride represented by a general formula of

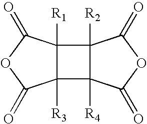

(in which formula, each of R₁, R₂, R₃, and R₄ is independently a hydrogen atom, a fluoro group, or an alkyl group with a carbon number of 1 or more and 4 or less.) or a tetracarboxylic dianhydride represented by a chemical formula of

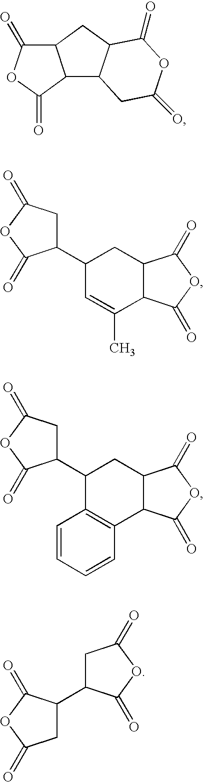

Embodiment (4) of the present invention is the polyamic acid as described in embodiment (3) above, characterized in that a molar ratio of the first diamine to a total amount of the diamines is 20% or more and 99% or less.

Embodiment (5) of the present invention is the polyamic acid as described in embodiment (3) or (4) above, characterized in that its number average molecular weight is 3×10³ or more and 5×10⁵ or less.

Embodiment (6) of the present invention is a polyimide characterized by being to be obtained by causing a dehydration and ring-closing reaction of the polyamic acid as described in any one of embodiments (2) to (5) above.

Embodiment (7) of the present invention is a laminated structure characterized by having a laminated structure wherein an electrically conductive layer on an ultraviolet-ray-irradiated area of a wettability changing layer containing a polyimide to be obtained by causing a dehydration and ring-closing reaction of the polyamic acid as described in any one of embodiments (3) to (5) above is formed on a substrate.

Embodiment (8) of the present invention is an electronic element array characterized by having the laminated structure as described in embodiment (7) above.

Embodiment (9) of the present invention is an image display medium characterized by having the electronic element array as described in embodiment (8) above.

Embodiment (10) of the present invention is an image display device characterized by having the image display medium as described in embodiment (9) above.

According to at least one embodiment of the present invention, it may be possible to provide a diamine having biodegradability and being capable of causing a ring-opening and addition polymerization reaction with a tetracarboxylic dianhydride in good yield, a polyamic acid to be obtained by causing a ring-opening and addition polymerization reaction of the diamine with a tetracarboxylic dianhydride, and a polyimide to be obtained by causing a dehydration and ring-closing reaction of the polyamic acid.

According to at least one embodiment of the present invention, it may also be possible to provide a laminated structure in which a wettability changing layer is formed which has biodegradability and allows for a reduction in ultraviolet ray irradiation for changing its surface free energy, and an electronic element array, image display medium and image display device having the laminated structure.

The present invention is not limited to any of the embodiment(s) and/or example(s) described above, and the embodiment(s) and/or example(s) may be altered, modified, or combined without departing from the spirit and scope of the present invention.

The present application claims the benefit of its priority based on Japanese Patent Application No. 2009-125634 filed on May 25, 2009 in Japan, the entire content of which is herein incorporated by reference.

What is claimed is:

1. A diamine represented by a general formula of: 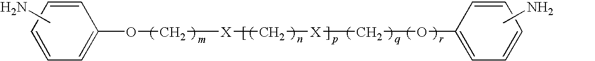
wherein X is an ester bond, each of m and n is independently a natural number, each of p and r is independently 0 or 1, q is an integer of 0 or more, a sum of m, n, and q is 20 or less, and when q is 0, p is 1 and r is 0.

2. A polyamic acid obtainable by a ring-opening and addition polymerization reaction of a diamine including the diamine as claimed in claim 1 and a tetracarboxylic dianhydride.

3. A polyamic acid obtainable by a ring-opening and addition polymerization reaction of diamines, including a first diamine and a second diamine, and a tetracarboxylic dianhydride, wherein:
the first diamine is the diamine as claimed in claim 1;
the second diamine is a diamine represented by a general formula of: 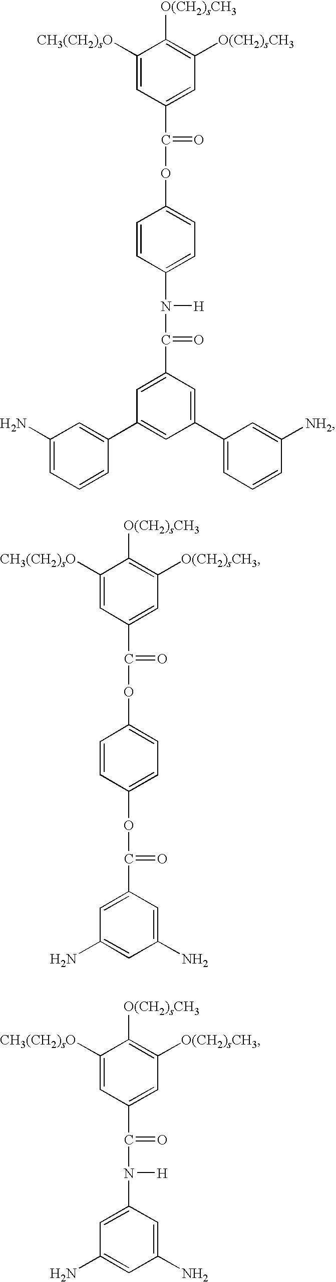 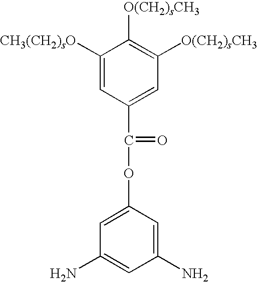
wherein s is an integer of 5 or more and 13 or less; and
the tetracarboxylic dianhydride includes:
a tetracarboxylic dianhydride represented by a general formula of: 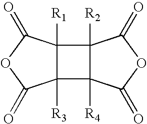
wherein each of R₁, R₂, R₃, and R₄ is independently a hydrogen atom, a fluoro group, or an alkyl group with a carbon number of 1 or more and 4 or less; or
a tetracarboxylic dianhydride represented by a chemical formula of:

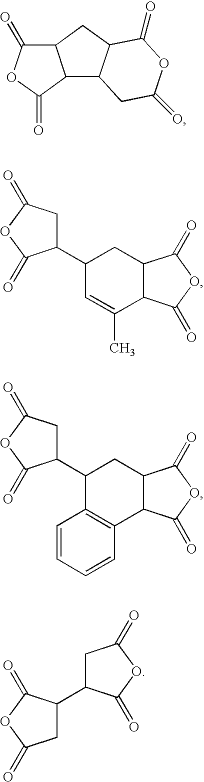

4. The polyamic acid as claimed in claim 3, wherein a molar ratio of the first diamine to a total amount of the diamines 20% or more and 99% or less.

5. The polyamic acid as claimed in claim 3, wherein a number average molecular weight thereof is 3×10³ or more and 5×10⁵ or less.

6. A polyimide obtainable by a dehydration and ring-closing reaction of the polyamic acid as claimed in claim 2.

7. A laminated structure comprising a laminated structure wherein an electrically conductive layer on an ultraviolet-ray-irradiated area of a wettability changing layer containing a polyimide obtainable by a dehydration and ring-closing reaction of the polyamic acid as claimed in claim 3 is formed on a substrate.

8. An electronic element array comprising the laminated structure as claimed in claim 7.

9. An image display medium comprising the electronic element array as claimed in claim 8.

10. An image display device comprising the image display medium as claimed in claim 9.
